(19) United States
(12) Patent Application Publication
(10) Pub. No.: US 2019/0180842 A1
(43) Pub. Date: Jun. 13, 2019

(54) GENETIC CUSTOMIZATION OF AN ORGANISM BASED UPON ENVIRONMENTAL PARAMETERS

(71) Applicant: Anthony LEONARDI, Bethesda, MD (US)

(72) Inventor: Anthony LEONARDI, Bethesda, MD (US)

(21) Appl. No.: 16/302,620

(22) PCT Filed: May 18, 2017
(86) PCT No.: PCT/US2017/033416

§ 371 Date: Nov. 16, 2018

Related U.S. Application Data
(60) Provisional application No. 62/338,165, filed on May 18, 2016.

(51) Int. Cl. G16B 20/00; C12Q 1/68; G16B 40/00; G06N 3/02; G16B 30/00

(52) U.S. Cl. CPC G16B 20/00; C12Q 1/68; G16B 30/00; G06N 3/02; G16B 40/00

(57) ABSTRACT

Genetic customization includes receiving a genotype of a species of interest, receiving a specification including an environmental parameter and/or (a) phenotype(s) of interest, and determining the genetic permutations most suitable for the environmental parameters and/or the phenotypes desired as listed by the recipient, based at least in part on different pairings of the genotype of the recipient and a genotype of a donor in the plurality of donors, and identifying a preferred strain/permutations species among the plurality of strains/species according to the chosen parameters, based in part on the percentage congruence determined.

### CROSS-REFERENCE TO RELATED APPLICATIONS

This application claims priority from U.S. Provisional Application Ser. No. 62/338,165 filed on May 18, 2016, which is incorporated herein by reference in its entirety.

### STATEMENT REGARDING FEDERALLY SPONSORED RESEARCH OR DEVELOPMENT

Not Applicable.

### INCORPORATION-BY-REFERENCE OF SEQUENCE LISTING

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### FIELD

The present invention relates to genetic customization related to an environmental parameter and/or phenotype of interest.

### INTRODUCTION

With the advent of de novo DNA/RNA synthesis, which allows for the creation of full genomes from scratch, the ability to customize, enhance, and optimize genomes and select phenotypes without the employment of arduous targeted mutagenesis techniques like CRISPR and retroviral targeting/shRNA becomes feasible. Current state of the art is the modification of a limited number of genes, or even a single gene for an embryo in a given species. For example, scientists have recently cured a patient of their sickle cell anemia by use of CRISPR to delete and replace the DNA responsible for the gene in the progenitor cells of the red blood cell.

Targeted mutagenesis via CRISPR and other methods are not limited to human therapies. Genomes of other organisms like bacteria and plants are modified to suit specific purposes. Bacteria can be used to clean oil spills. Plants can be cross-bred to have desirable traits in progeny, or have specific sites mutated.

Therefore, what is needed are systems and methods for customizing genomes, or genetic customization, related to an environmental parameter and/or phenotype of interest.

### SUMMARY

The present teachings include systems for genetic customization including one or more processors. The processors are configured to receive a specification including an environmental parameter or phenotype of interest; receive a genotype of an organism to be modified; determine genetic permutations pertaining to the phenotype of interest based at least in part on optimal fitness according to environmental parameters, recipient specifications, and loci of disease; and deliver the optimized genotype to the recipient. The system also includes memory coupled to the processor, configured to provide the processor with instructions.

In accordance with a further aspect, the system can include a database coupled to the processor, configured to store the genetic permutations. In yet another aspect, determining the fitness level of a given genotype includes determining all possible genomes and applying them to selected environmental parameters and specifications, which may or may include vectors considering traits that may cause illness. In yet another aspect, determining the fitness level of a given genotype includes applying all possible combinations of genetic permutations to recipient selected environmental parameters and specifications, which may include traits that may cause illness.

In accordance with yet another aspect, the processor is further configured to identify a strain among the plurality of strains most congruent with the recipient specifications, based at least in part on the percentage match determined. In another aspect, the specification includes a plurality of independent environmental parameters.

In accordance with another aspect, the processor is further configured to identify any strain that is deemed to be a close percentage match of the optimized genome. In yet another aspect, the processor is further configured to receive an additional specification or specifications that includes an additional phenotype of interest; and determine additional genetic permutations pertaining to the additional phenotype of interest.

In accordance with yet another aspect, the processor is further configured to supply display information for displaying the additional genetic permutations relevant to the phenotype(s) of interest.

The present teachings also include methods for genetic customization, the method comprising receiving a specification including an environmental parameter or phenotype of interest; receiving a genotype of an organism to be modified; calculating fitness pertaining to selected environmental parameters, recipient preference, and loci of disease; determining genetic permutations pertaining to the phenotype of interest based at least in part on optimal fitness according to environmental parameters, recipient specifications, and loci of disease; and delivering the optimized genotype to the recipient.

In accordance with a further aspect, the method can include storing the genetic permutations of the plurality of environmental parameters, recipient specifications, and loci of disease. In yet another aspect, determining the genetic permutations pertaining to the optimized genome is further based on calculation of fitness. In yet another aspect, determining the fitness level of a given organism includes determining all possible combinations of genetic permutations.

In a further aspect, the recipient specifications are any combination of environmental parameters, phenotypes, and loci of disease, and determining the optimal genome accounts for the specifications.

In yet another aspect, the method can further comprise excluding any donor that is deemed to be a close relative of the recipient.

In a further aspect, the method can further comprise receiving an additional specification that includes an additional phenotype of interest; and determining additional statistical information pertaining to the additional phenotype of interest based at least in part on an additional genotype of the recipient and an additional genotype of the preferred donor.

In yet another aspect, determining the genetic permutations pertaining to the optimized genome is further based on calculation of fitness is done by use of a neural network.

The present teachings also include a computer program product for genetic customization, the computer program product being embodied in a computer readable storage medium and comprising computer instructions for receiving a specification including a phenotype of interest; receiving a genotype of an organism and a plurality of genotypes of existing species; determining fitness levels of all possible genomes based at least in part on recipient specifications including environmental parameters, loci of disease, and recipient phenotype preferences; and determining percentage congruence pertaining to the genotype of interest vs. existing strains based on environmental parameters and/or recipient specifications.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1 is a block diagram illustrating an embodiment of a genome optimization system.

FIG. 2A is a flowchart illustrating an embodiment of a process for optimization of a given genome.

FIG. 2B is a flowchart illustrating an embodiment of a process for providing best-suited environment information to the recipient.

FIG. 2C is a flowchart illustrating an embodiment of a process for providing existing organism strain information including the best suited environment to the recipient.

FIG. 3 is a diagram illustrating an embodiment of a recipient interface for making recipient specification and displaying the results.

FIG. 4 is a diagram illustrating an embodiment of a recipient interface for making recipient specification and displaying the results of a best-suited strain.

FIG. 5 is a diagram illustrating an embodiment of a recipient interface that allows the recipient to view additional traits and environmental parameters suited to the organism.

### DETAILED DESCRIPTION

Abbreviations and Definitions

To facilitate understanding of the invention, a number of terms and abbreviations as used herein are defined below as follows:

As used herein, a phenotype refers to a certain characteristic or trait of an organism, such as morphological, developmental, biochemical, physiological, or behavioral properties. Height, pest resistances, eye color, size, gender, personality characteristics and risk of developing certain types of cancer are examples of phenotypes. As used herein, genotype refers to information pertaining to the genetic constitution of a cell, an organism, or an individual in reference to a specific character under consideration, for example, information pertaining to a combination of alleles located on homologous chromosomes that is associated with a specific characteristic or trait.

As used herein, a genetic permutation refers to any single genetic trait pertaining to the genetic constitution of a cell, an organism, or an individual in reference to a specific character under consideration, for example, information pertaining to a combination of alleles located on homologous chromosomes that is associated with a specific characteristic or trait. It is interchangeable with the term marker.

As used herein, a genome refers to a list of genetic permutations, a list of genotypes, and/or a list of all the genetic information pertaining to an organism. As such, genomic, as used herein, would refer to this definition of genome.

As used herein, a loci of disease refers to any genetic permutation which confers any illness, disease, infection, increased likelihood of illness, disease, or infection, or fitness disadvantage to an organism regardless of environmental parameter. These genetic permutations labeled as loci of disease are not included in the product generated from the optimization process and, if they exist in the genome to be optimized, they are removed.

As used herein, a relative fitness value refers to a numerical value assigned to a particular genetic permutation in a given environment depending on the fitness gain or loss it confers to an organism within that specific environment.

As used herein, an environmental parameter is an attribute of the environment with a qualitative or quantitative specification. For example, temperature, PH, acidity, salinity, oxygen level, carbon dioxide level, gravity, sunlight, and humidity are all environmental parameters which are relevant to many organisms. The list of environmental parameters here is not exhaustive, and the scope includes but is not limited to anything the organism is subject to or may be subject to.

As used herein, a recipient is an entity which provides specifications for the optimization of an organism. A recipient is also an entity which receives the optimized genome/list of genetic permutations.

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application. In the following description, it is understood that terms such as “first,” “second,” “top,” “bottom,” “up,” “down,” and the like, are words of convenience and are not to be construed as limiting terms.

Genetic Customization of an Organism Based Upon Environmental Parameters and/or Recipient Preference

While the identification and replacement of a single polymorphism is ambitious, this state of the art will be left behind in favor of full DNA sequence printing, with which DNA an organism may be modified or even fully engineered. Given this coming advancement, scientists and individuals will be able to modify the genomes of organisms on a much larger scale than one, two, or a handful of genetic attributes at a time. In such a scenario where genomes may be synthesized, retaining SNPs or genetic permutations in an organism which may cause illness or not be conducive to the environment is an avoidable disadvantage. Since genomes can be customized, there is a need and an opportunity for the optimization of genetic attributes. It would be reasonable and necessary to customize organisms according to the environment which they would exist in.

The genetic customization of an organism allows for the creation of an organism suited to a specific environment, thereby bypassing the process of natural selection wherein environmental pressures exerted on members of a species over time (generations) allow for greater fitness specific to said environment. The ability to customize genomes will be of use to those who need organisms like food plants and bacteria best suited phenotypically for specific tasks, specific environments, and to the recipient's preferences. Additionally, it would allow for the creation of animals (like humans) without susceptibility to diseases caused by or associated with given genetic permutations.

In situations where the environment may be unpredictable, or have parameters that are not typical for organisms to exist in, the in silica generation of organisms best suited for that environment would bypass the need for piecemeal and/or gradual adaptation via natural selection. With an in silica process of selection of the most optimal genetic permutations which confer the highest level of fitness for the environmental qualities the organism would be subjected to, the generation of an organism which specifies multitudes of permutations best suited for an environment can be achieved.

Unlike US Patent Application No. 20100145981, this patent does not use statistical calculations to approximate the likelihood of given phenotypes or genetic markers of inheritance. Instead, the genetic permutations placed in the genome (SNPs, etc.) are determined by the environmental parameters indicated by the recipient, or by the recipient's desire for a genetic trait which is associated with a phenotypic outcome. As such, statistical models designed to reveal the likelihood of a given outcome for the progeny are unnecessary due to the direct identification and determination of the genetic permutations to be included. This process constitutes the optimization of a genome, rather than its prediction.

Additionally, a mathematical process where weights are assigned to genetic permutations in terms of the fitness advantage they confer to specific environments allows all possibilities of permutations to be considered and ranked, according to a relative fitness value. This system also allows for the incorporation of recipient phenotypic mandates, by assigning a large numerical value to the specific qualities that are picked by the recipient.

Genome Optimization

Genetic customization of an organism based upon environmental parameters and/or recipient preference is described. The organism's genetic information such as genome sequences, genetic permutations, and/or marker information is obtained and stored. In some embodiments, the recipient is allowed to make a specification of one or more phenotypes of interest in the product and make a specification of one or more environmental parameters the product is to be optimized for. In some embodiments, all combinations of genetic permutations comprising the product are determined, and the product with the highest score of fitness is delivered to the recipient. In some embodiments, the list of genetic permutations and/or novel genetic sequence is determined, based on the environmental parameters and/or recipient specifications provided by the recipient. Additionally, there may be a mathematical comparison to determine percentage congruence between the marker information of the optimal product and the marker information of existing strains. Based on the percentage congruence, existing strains are presented to the recipient. In some embodiments, the recipient is allowed to view further phenotypic details about a selected strain of interest.

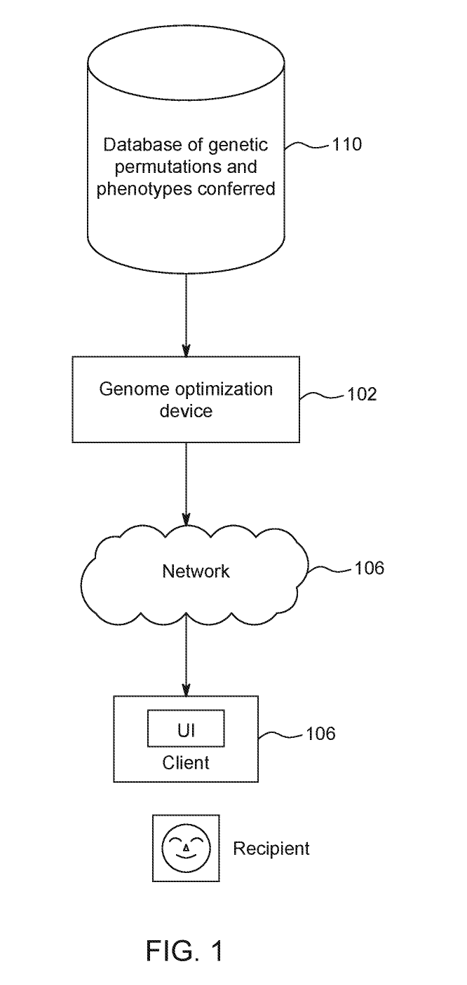

FIG. 1 is a block diagram illustrating an embodiment of a donor selection system. Genome Optimization Device 102 may be implemented using a server computer with one or more processors, a stand-alone computing device such as a desktop computer, a mobile device, specialized hardware device designed for implementing the donor selection process, or any other appropriate hardware, software, or combinations thereof. The operations of the donor selection device are described in greater detail below. In this example, some recipient such as the agent, customer, or a system operator, accesses the donor selection device via a network 106 using a client device 108 that provides a genome optimization recipient interface 104. Alternatively, the recipient can access the genome optimization device directly, for example by using software executing on the donor selection device, without requiring communication over a network. Information (including genetic information and optionally other information such as environmental data and phenotypes pertaining to the potential genetic permutations) is stored in a database 110, which can be implemented on an integral storage component of the donor selection device, an attached storage device, a separate storage device accessible by the donor selection device, or a combination thereof. Many different arrangements of the physical components are possible in various embodiments. In various embodiments, the entire genome sequences and/or specific markers (e.g., Single-Nucleotide Polymorphisms (SNPs), which are points along the genome with two or more common variations, Copy-Number Variations (CNVs), which are inserted or deleted lengths of DNA, etc.) are stored in the database to genome optimization.

The recipient may specify certain phenotypes the recipient desires in his/her hypothetical product and send the specification to the genome optimization device. As will be described in greater detail below, based on the genotype information of the chosen species/or genome provided, the genome optimization device selects the relevant genetic permutation(s) pertaining to the phenotype(s) of interest and presents the result to the recipient.

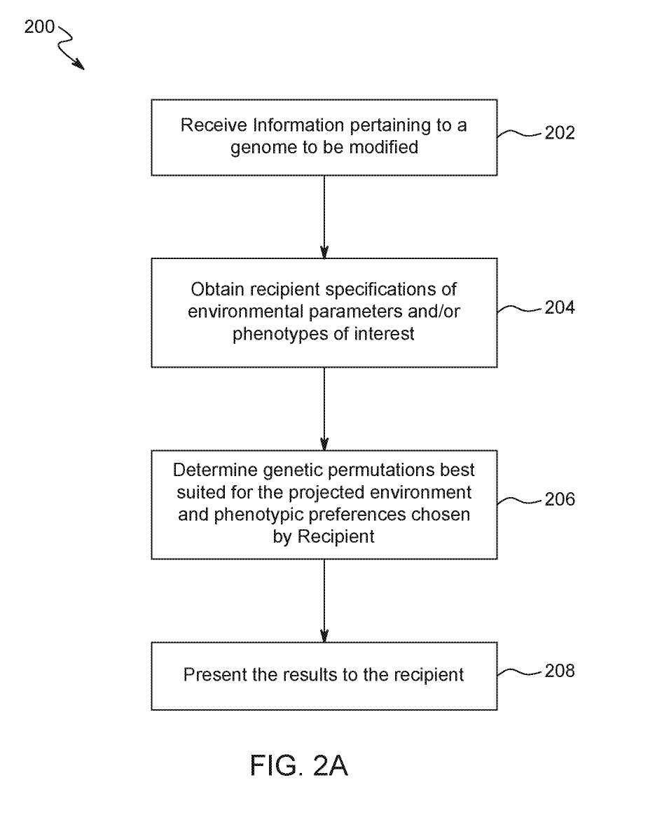

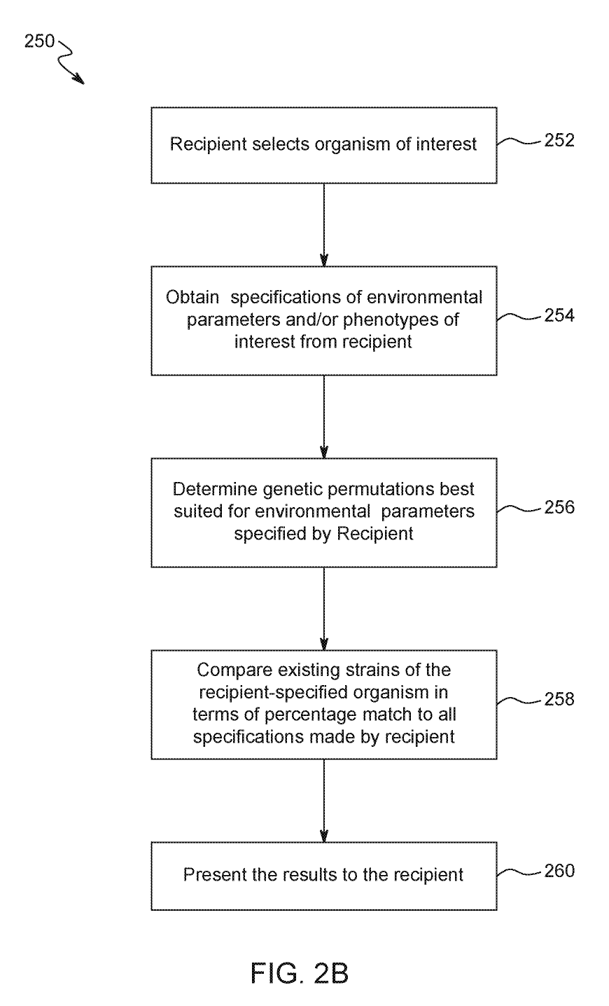

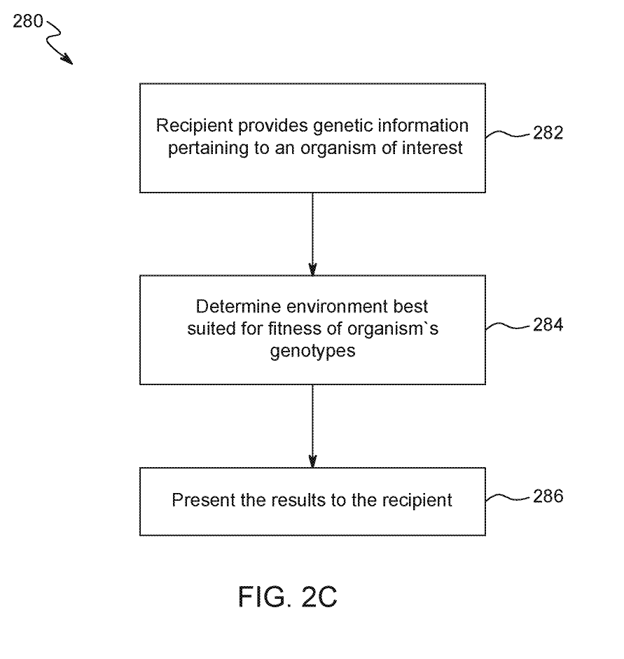

FIG. 2A is a flowchart illustrating an embodiment of a process for optimization of a chosen genome. Process 200 may be implemented on a genome optimization device such as 102 of FIG. 1. The process initiates at 202. The genotype of the organism to be modified is received. The genotype information is received from a database in this example. In some embodiments, the genotype is received in a batch. In some embodiments, the genotype is received in multiple steps individually or in groups. In some embodiments, a genotype is not provided, but rather a species is selected which has a genotype stored in a database.

At 204, specifications including environmental parameters and/or one or more phenotypes in a hypothetical organism is received from a recipient. In some embodiments, the system is configured with a set of available phenotypes and/or combination of phenotypes from which the recipient selects the set of phenotypes of interest. Various types of environmental parameters (e.g., soil PH, sunlight levels, oxygen levels), phenotypes such as physical traits (e.g., height, weight, growth under certain eye color, etc.), and inherited diseases (e.g., certain types of cancer, congenital heart defects, deafness, etc.) are provided through a recipient-selectable interface. The recipient's selection forms the specification. For example, the recipient may use recipient interface tools such as selection boxes to indicate that he/she desires an organism that is best suited for a given PH, given temperature, and has a large food product. In some embodiments, the recipient is allowed to form a qualitative query in natural language, such as “genetic permutations in this database would be most likely to yield the sweetest fruit, with the lowest susceptibility to aphids, and to have less than a 0.01% chance of not bearing fruit?” The natural language query is parsed to form the specification. In some embodiments, the recipient may also express his or her preferences, for a human example “I prefer low risk of colorectal cancer and congenital heart defects and I prefer green eyes to other colors. The risks of colorectal cancer and heart defects are equally important to me, and are more important to me than eye color. What are the most suitable genetic permutations in this database, subject to these preferences?” In some embodiments, the recipient is allowed to make a general specification such as the longest expected life span, a soil pH of 7.2, a high Nitrogen content in the soil, a certain temperature of the ambient ocean water, the expected lifetime cost of healthcare, the expected lifetime cumulative duration of hospitalization, etc. The general specification is implemented as a combination of various specific phenotypes in some embodiments and as a single genotype influenced by multiple genotypes in other embodiments. In some embodiments, certain groups of phenotypes are placed into a single option.

At 206, the genetic permutation(s) corresponding to the chosen environmental parameters and phenotypes are determined based on the genome/species provided/selected. A phenotype may be affected by one or more markers in the genome. A specific environment may require a certain genetic permutation, or combination of permutations. For example, if the specification of a phenotype is the longest expected life span, then markers/genetic permutations known to be associated with longevity (based on previous studies, etc.) may be employed; if the specification is the least expected lifetime cost of health care, then markers known to be associated with chronic diseases and/or diseases with expensive treatments may be employed; if the specification is the least expected lifetime cumulative duration of hospitalization, then markers known to be associated with diseases requiring hospitalization may be employed. In some embodiments, a neural network is used to determine the relative fitness values specific to genetic permutations in specific environments. In some embodiments, the phenotypes specified by the recipient are not independent. For example, skin color and hair color are non-independent phenotypes. Non-independent phenotypes may occur because the phenotypes are influenced, at least in part, by the same genetic marker or markers. Non-independence may also occur because the phenotypes depend on genetic markers that are located near one another in the genome. Non-independence, such as that between height and weight, may also be caused by non-genetic factors, such as developmental or environmental factors that influence the phenotypes separately from the genotype or in interaction with the genotype. In some embodiments, certain genetic permutations that care known to be deleterious are identified and nullified and/or replaced.

At 208, The results of the genome optimization process are presented to the recipient. In some embodiments, these are as a list of the genetic permutations/markers responsible for each desired quality. In other embodiments, the result is given as a full genome which can be viewed or downloaded.

FIG. 2B is a flowchart illustrating an embodiment of a process similar to 2A, but additionally presenting the recipient with existing strains of organisms that closely match the desired specifications the recipient has input. In some embodiments, process 258 may be inserted, removed, or the sole outcome. It may be implemented on a donor selection device such as 102 of FIG. 1. The process starts at 252, where information pertaining to a selected organism, such as a specific genomic sequence, genetic sequence, list of genetic permutations, or selected species, is received. In some embodiments, the organism may be selected by the recipient from a list of organisms. In some embodiments, a specific sequence is provided by the recipient. In some embodiments, a list of genetic permutations is provided by the recipient. In some embodiments, the system provides genetic or genomic information for this process. At 254, another specification including one or more additional phenotypes and/or environmental parameters of interest Is/are obtained. In some embodiments, the recipient inputs the specification in a way similar to 202 of process 200. In some embodiments, certain phenotypes and parameters are preconfigured by the system as environments and/or phenotypes that may be of interest to the recipient. In some embodiments, the information was already previously obtained during process 200, and this step is therefore omitted. At 258, percentage congruence between the desired product according to the recipient's specifications and existing strains is determined based on the genotype information of the optimized product and the genotype information of the existing strains. At 260, the results are presented to the recipient.

The recipient may repeat process 200, process 250, or both to find a most suitable product.

FIG. 2C is a flowchart illustrating an embodiment of a process for disclosing the most suitable environment for a given genome or strain to the recipient. In process 282, a genome is provided by the recipient. In some embodiments, genetic markers and their permutations are provided instead of a full genome. In some embodiments, the strain is selected from a list. At process 284, the optimal environment given each genetic permutation is calculated/retrieved from the database. At process 286, this information is presented to the recipient.

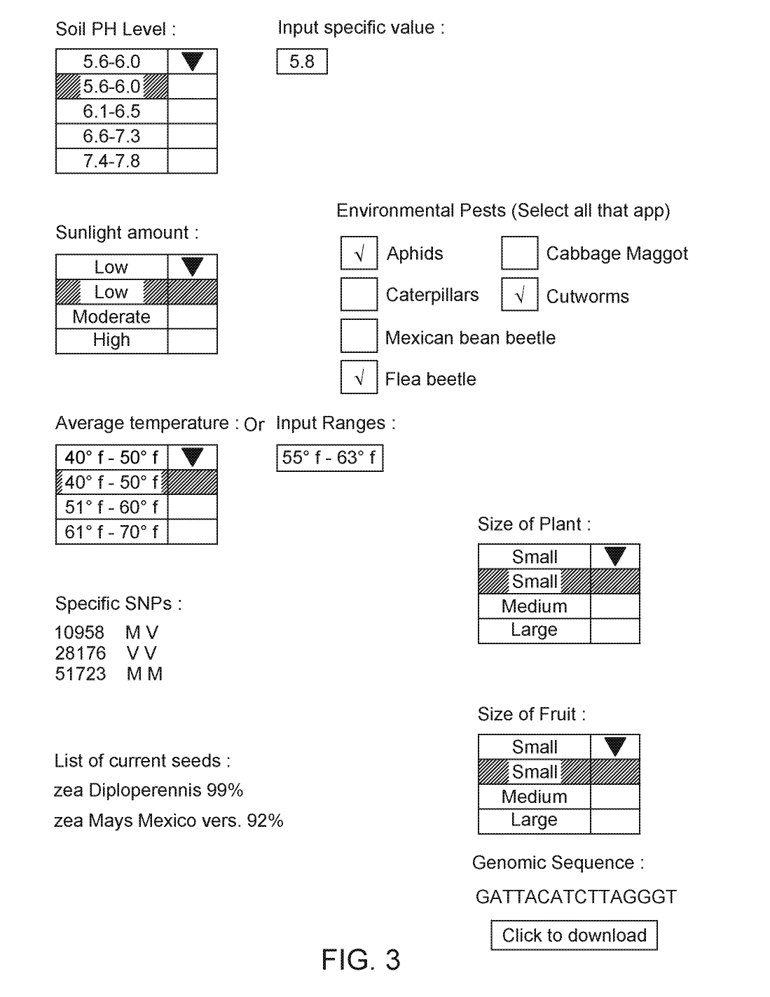

FIG. 3 is a diagram illustrating an embodiment of a recipient interface for making recipient specification and displaying the results. In this example, the recipient has specified that she prefers an organism suited for the PH range 5.6-6.0, and has specifically put in the value of 5.8. She has also indicated they would like the organism to be suited to low sunlight, and resistance to the pests including aphids, cutworms, and flea beetles. Finally, she would like the organism to thrive at a 55 to 63 degree Fahrenheit temperature, a small plant size, and a small fruit size. A genetic permutation optimization process such as 200 is performed, and the results page shows the list of necessary genetic permutations, the genetic information of the organism with these specifications, and the percentage congruence of the ideal sequence with the qualities of existing strains. Alternatively, all the existing strains can be shown in a ranked list. Additionally, there may be presented an option to download the novel genome or list of genetic permutations.

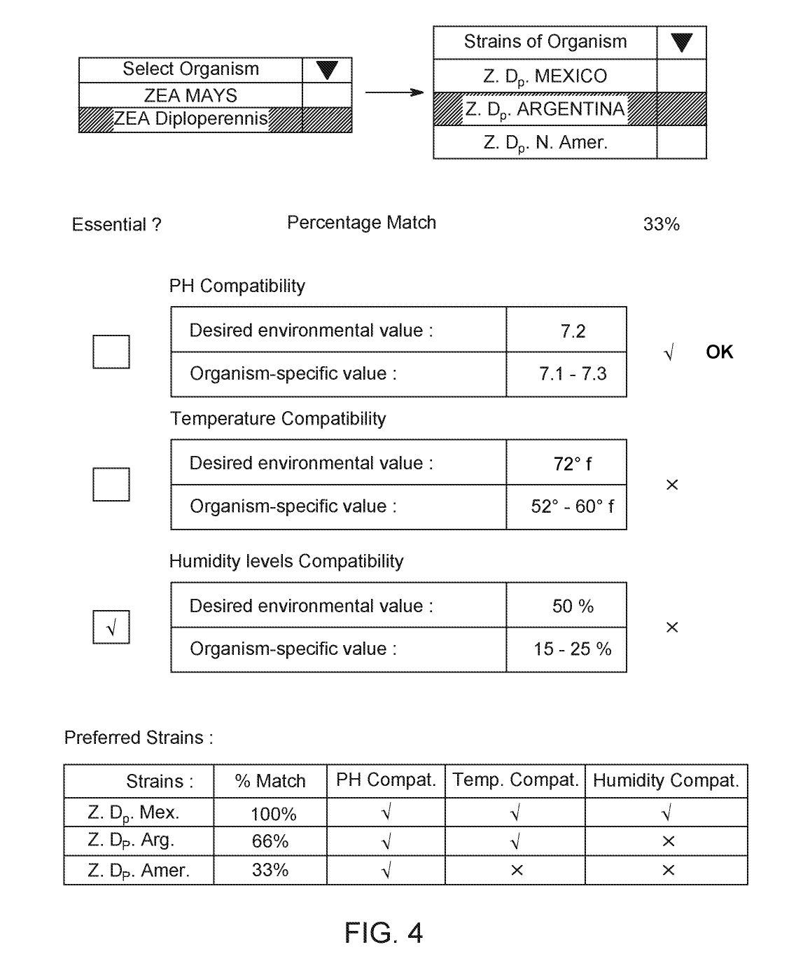

FIG. 4 is a diagram illustrating an embodiment of a recipient interface for selecting a species and strain of interest and displaying the results pertaining to existing strains and one best optimized for the environmental parameters and/or desired attributes. In this example, the recipient has selected the Argentinian strain of ZEA Diploperennis to optimize. They have input the environmental parameters of a soil PH of 7.2, an ambient temperature of 72 degrees Fahrenheit, and a 50% humidity level. The recipient has also indicated that the compatibility for the humidity level of 50% is essential in the strains presented. A donor selection process such as 200 is performed and the genome optimization device is presenting in the recipient interface the strain Z. DP. Mex. As the only compatible strain, with a 100% match and a compatible humidity range. Alternatively, all the donors can be shown in a ranked list.

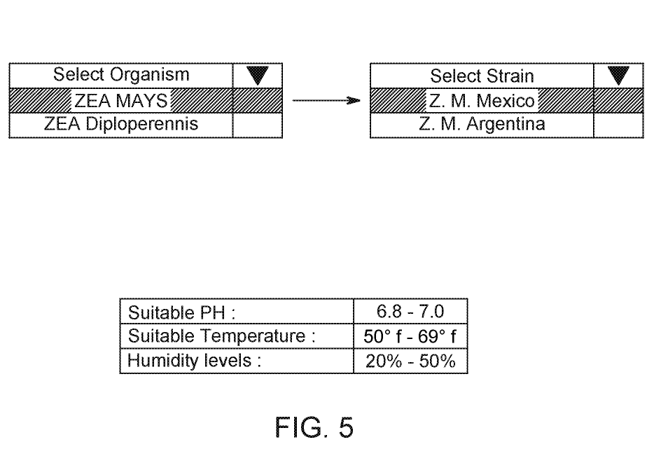

FIG. 5 is a recipient interface diagram illustrating another embodiment of a recipient interface that displays the optimal environment for a given species and strain. In this example, the recipient has specified they would like to see the preferred environment of the Zea Mays strain from Mexico. The results page shows the suitable PH range, the suitable temperature range, and the suitable humidity levels.

Genetic customization based on environmental parameters and recipient preference has been disclosed. The technique allows the recipient to have a customized organism that is best suited to its prospective environment and recipient preferences.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

The systems and methods disclosed herein may be implemented via one or more components, systems, servers, appliances, other subcomponents, or distributed between such elements. When implemented as a system, such systems may include an/or involve, inter alia, components such as software modules, general-purpose CPU, RAM, etc., found in general-purpose computers. In implementations where the innovations reside on a server, such a server may include or involve components such as CPU, RAM, etc., such as those found in general-purpose computers.

Additionally, the systems and methods herein may be achieved via implementations with disparate or entirely different software, hardware and/or firmware components, beyond that set forth above. With regard to such other components (e.g., software, processing components, etc.) and/or computer-readable media associated with or embodying the present implementations, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the systems and methods may be achieved via or performed by logic and/or logic instructions including program modules, executed in association with such components or circuitry, for example. In general, program modules may include routines, programs, objects, components, data structures, etc., that perform particular tasks or implement particular instructions herein. The embodiments may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, where media of any type herein does not include transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the implementations described herein or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the implementations herein, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media.

In further embodiments, a machine learning algorithm is used to observe growth of organisms via the collection of data from primary and/or secondary sources, and modify the fitness values assigned to specific genetic permutations to reflect the observations and/or recordings.

Moreover, the above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

### EXAMPLES

Aspects of the present teachings may be further understood in light of the following examples, which should not be construed as limiting the scope of the present teachings in any way.

### Example 1

In this example, a human genome is optimized based on three different mechanisms. 1) recipient parameters for the organism to be best suited for, 2) a phenotypic preference indicated by the recipient, and 3) markers associated with disease identified and replaced by the program. In this example, the recipient has provided a list of human Genetic markers to be optimized. That list includes the following markers: rs12097901 (G;G), rs1815739 (C;C), rs1121923 (C;C), rs148833559 (A;C), rs1042725 (C;C), rs17822931 (C;C), i3003626 (I;I), rs429358 (C;T), and rs7412 (C;C)

In this example, our recipient would like to have a human organism that is suited for space travel and exploration of unfamiliar terrain under possible high gravity. Food sources would be heavy in fats and oils as they are calorific, oxygen may be in limited supply, and quarters with other humans may be tight.

So the environmental parameter are chosen as:
Endurance=true, Hypoxia=True, High Fat Diet=True

And the phenotypic parameters are chosen as:
Average Height=True, No Body Odor=true

This is queried against the database where the genetic permutations are chosen according to the values given by the recipient, shown in the following tables:

TABLE 1
Optimization to environment

| | Recipient | Variation 1 | Variation 2 |
|---|---|---|---|
| Marker | Genotype | Allele | Allele |
| rs1815739 | C; C | Endurance = true | Strength/ |
| | | T; T | Sprint = true C; C |
| rs12097901 | G; G | Hypoxia = true | Normoxia = true |
| | | C; C | G; G |
| rs1121923 | C; C | High Fat | Low fat |
| | | Diet = true T; T | diet = true C; C |

Table 1 represents a database of permutations to be selected according to values indicated by the recipient. Cross-reference of the provided markers with the preference indicated by the recipient allows the adjustment to: rs12097901 (C;C), rs1815739 (T;T), rs1121923 (T;T)

TABLE 2
Phenotypic Preference

| | Recipient | Variation 1 | Variation 2 | Variation 3 |
|---|---|---|---|---|
| Marker | Genotype | Allele | Allele | Allele |
| rs148833559 | A; C | Taller = | Average | |
| | | true A; C | height = | |
| | | | True C; C | |
| rs1042725 | C; C | .8 cm | .4 cm | Average |
| | | Taller = | Taller = | height = |
| | | true C; C | true G; C | true G; G |
| rs17822931 | C; T | normal | Balanced = | No Body |
| | | colostrum = | true C; T | odor = |
| | | true T; T | | true C; C |

Table 2 represents a database of permutations to be selected according to phenotypic preferences indicated by the recipient. Cross-reference of the provided markers with the preference indicated by the recipient allows the adjustment to: rs148833559 (C;C), rs1042725 (G;G), rs17822931 (C;C),

TABLE 3
Disease Markers

| | Recipient | Variation 1 |
|---|---|---|
| Marker | Genotype | Allele |
| i3003626 | I; I | HIV resistance = true D; D |
| rs429358 | C; T | Alzheimer's reduced risk = true T; T |
| rs7412 | C; C | Alzheimer's reduced risk = true T; T |

Table 3 is a database used to remove disease-associated permutations. Cross-reference of the provided markers with the preference indicated by the recipient allows the adjustment to: i3003626 (D;D), rs429358 (T;T), and rs7412 (T;T)

This makes the final list of the optimized permutations as: rs12097901 (C;C), rs1815739 (T;T), rs1121923 (T;T), rs148833559 (C;C), rs1042725 (G;G), rs17822931 (C;C), i3003626 (D;D), rs429358 (T;T), and rs7412 (T;T). This list is presented to the recipient. In some embodiments the recipient may choose to download this list or view it, or download a genome with these changes incorporated.

In some embodiments, any combination of one or all three types of adjustment to the genome (recipient preference vs. environmental parameters vs. disease associated marker removal) may be employed, either to the full genome pertaining to an organism or species or a list of markers pertaining to an organism or species.

### Example 2

In this example, a human genome is optimized according to phenotypic preference, fitness within the environment, and to remove a disease marker. This is achieved with assigned fitness values for each genetic permutation in the following method.

An equation which can be used to describe the calculation of fitness is:

$$F = {\sum }_{x = 1}^{n} {Γ}_{x} \cdot {ɛ}_{x}$$

The recipient provides a genome with the following:
α1 rs12097901 (G;G) (this genotype is represented as the vector α1 (1, 0, 0)
(If it were (G;C) it would be α1 (0, 1, 0), and if it were (C;C) it would be α1 (0, 0, 1))
α2 rs148833559 (C;C) (this genotype is represented as the vector α2 (0, 0, 1)
(If it were (A;A) it would be α1 (1, 0, 0), and if it were (A;C) it would be α1 (0, 1, 0))
α3 rs7412 (C;T) (this genotype is represented as the vector α3 (0, 1, 0) (If it were (C;C) it would be α1 (1, 0, 0), and if it were (T;T) it would be α1 (0, 0, 1))

This series of markers is then concatenated into the vector: Γ<α1, α2, α3>which is notated as Γ1<1, 0, 0, 0, 0, 1, 0, 1, 0>

In some embodiments, there are more than three genotypes which would make Γ<α1, α2, α3, α4, . . . > and in some embodiments, there are less than three genotypes.

In some embodiments, a neural network is used to generate fitness values for given genetic permutations in specific environments. A neural network can be used to find the genetic permutations best suited for the environment and/or fitness of the organism.

The gamma vector, which is specific to the genotype listed by the recipient, is then applied to a vector which has assigned values for the fitness of each genotype within each specific environmental parameter and preference parameter listed by the recipient with the following equation:

Γ·εsum=F for Genetics×Environment=Fitness

To find εsum, which is the sum of fitness values given for all parameters including recipient specified and non-specified environments, we have a set of premade weights for each specific environment and its genetic permutation.

εsum=ε1+ε2+ε3 . . .

Therefore εsum is a composite function of previously derived individual εN values (i.e. ε1, ε2, ε3, etc.) In some embodiments there are more vectors of εN values and in some embodiments, there are fewer.

In this example, ε1 is the vector denoting the fitness values for each genetic permutation in a high altitude environment, ε2 is the vector denoting fitness values for each genetic permutation according to the recipient's desire for tallness in the product sequence, and ε3 is the vector denoting fitness values for specific permutations which may cause disease.

For example, for the trait rs12097901 (G;G), which denotes normal breathing at sea level instead of the variant (C;C) which allows for breathing at high altitude there is the following vector when a high altitude environment is selected by the recipient:

High altitude selectedε1<−10,0,10,−5,0,1,0,0,0>

ε1 has specific values corresponding to each genetic variation. Here, we show that high altitude fitness is not only affected by the three genetic variations specific to rs12097901, but that there is a disadvantage for tall individuals within the high altitude environment as well, shown with the −5 value for those individuals with rs148833559 (A;A), a 0 value for those with (A;C), and a 1 value for (C;C). The locus rs7412 which confers a risk for Alzheimer's does not have an associated fitness change/value with this environment, but will be considered when being cross-referenced with ε3 to make a final fitness value.

Had normal altitude been specified by the recipient instead of high altitude, a vector which is specific to normal altitude would be used for optimization as the ε1. Such a vector would have lower fitness values pertaining to the genetic permutation associated with an adaptation to high altitude respiration.

Next, our recipient has indicated they would like a tall organism as the product, so we have the attribute ε2 as:

ε2<0,0,1,100,50,0,0,0,0>

where we see a slight fitness advantage to people who are rs12097901 C;C denoted by the 1, we have a value of 100 for rs148833559 (A;A) (since this is the genotype conferring height to the recipient's product, and (A;T) also confers some height, so it is given the value 50, and T;T confers no additional height, so does not add to the fitness value. rs7412 has no known fitness conferrence to height so it has zero values for homozygosities and heterozygosity.

Next, ε3 accounts for which traits may cause illness, or in other embodiments, are recipient specifications for avoiding traits which cause illness. In these scenarios, they also have fitness vectors applied to the genome vector.

ε3<1,0,−1,−1,0,0,−1,50,100>

Here, we see homozygosity in rs12097901 (G;G) conferring an advantage where being homozygous in (C;C) confers a lower fitness. However, this is ultimately compensated for considering the product will experience a fitness advantage according to ε1 in the hypoxic environment. We also see the homozygous trait that confers height has a small fitness disadvantage. Finally we see a massive fitness advantage for the permutations which confer resistance to Alzheimer's Disease.

In some embodiments, the values for a specific genetic permutation are taken from the growth curve Y=Ae^{kx}, where the exponential value of proliferation in a given environment for an organism with given genetic permutations, is used as the values in the environmental vectors.

To find εsum, we add the environmental fitness vectors ε1, ε2, and ε3. In some embodiments, they can be multiplied, and in some embodiments, there are more and/or less than three ε vectors. Additionally, they may each be multiplied against Γ1 and then added in some embodiments, considering x(y+z)=(xy)+(x·z):

ε1<−10,0,10,−5,0,1,0,0,0>+ε2<0,0,1,100,50,0,0,0,0>+ε3<1,0,−1,−1,0,0,−1,50,100>=εsum<−9,0,10,94,50,1,−1,50,100>

so we have F1=Γ1·<−9,0,10,94,50,1,−1,50,100>

F1=Γ1<1,0,0,0,0,1,0,1,0,>·<−9,0,10,94,50,1,−1,50,100>

which make the value F1=(1x−9)+(1×1)+(1×50)=42

F1=42

This is the fitness value assigned to the current genome. The computer must generate every possible combination of genetic permutations and then cross-reference them to εsum to generate a rank of Γ vectors. In some embodiments, this step may be taken first.

In this example the processor generates 3³ genetic combinations and plugs them each into the equation Γ·εsum=F to find F2, F3, F4, and so on, until there is a list of fitness values F for each genome vector Γ:

Γ1<1,0,0,0,0,1,0,1,0>·εsum=42

Γ2<1,0,0,1,0,0,1,0,0>·εsum=84

Γ3<1,0,0,1,0,0,0,1,0>·εsum=135

Γ<1,0,0,1,0,0,0,0,1>·εsum=101

Γ5<1,0,0,0,1,0,1,0,0>·εsum=40

Γ6<1,0,0,0,1,0,0,1,0>·εsum=91

Γ7<1,0,0,0,1,0,0,0,1>·εsum=141

Γ8<1,0,0,0,0,1,1,0,0>·εsum=−8

Γ9<1,0,0,0,0,1,0,0,1>·εsum=92

Γ10<0,1,0,1,0,0,1,0,0>·εsum=93

Γ<0,1,0,1,0,0,0,1,0>·εsum=144

Γ12<0,1,0,1,0,0,0,0,1>·εsum=194

Γ13<0,1,0,0,1,0,1,0,0>·εsum=49

Γ14<0,1,0,0,1,0,0,1,0>·εsum=100

Γ15<0,1,0,0,1,0,0,0,1>·εsum=150

Γ16<0,1,0,0,0,1,1,0,0>·εsum=0

Γ17<0,1,0,0,0,1,0,1,0>·εsum=51

Γ18<0,1,0,0,0,1,0,0,1>·εsum=101

Γ19<0,0,1,1,0,0,1,0,0>·εsum=103

Γ20<0,0,1,1,0,0,0,1,0>·εsum=154

Γ21<0,0,1,1,0,0,0,0,1>·εsum=204

Γ22<0,0,1,0,1,0,1,0,0>·εsum=59

Γ23<0,0,1,0,1,0,0,1,0>·εsum=110

Γ24<0,0,1,0,1,0,0,0,1>·εsum=160

Γ25<0,0,1,0,0,1,1,0,0>·εsum=10

Γ26<0,0,1,0,0,1,0,1,0>·εsum=61

Γ27<0,0,1,0,0,1,0,0,1>·εsum=111

These are then ranked and the result with the highest numerical value for fitness is then delivered to the recipient. In some embodiments, it is as a list of genetic permutations, and in some embodiments, it is incorporated into a genome of interest. In this example Γ21 has the highest fitness value, and is deemed the optimal genome for the environment and is presented to the recipient where:

Γ21<0,0,1,1,0,0,0,0,1>is converted to rs12097901(C;C),rs148833559(A;A),rs7412(T;T)

### Example 3

In this example, a hypothetical bacterial genome is provided by the recipient for optimization. It is optimized according to phenotypic preference, fitness within the environment, and resistance to certain antibiotics. This is achieved with assigned fitness values for each genetic permutation in the following method.

The recipient provides an Oceanobacillus iheyensis genome with the FASTA format:
actttcaaaAaaatcagcgTaaaaaacatActaatttgggcaaattcccacctgtttttag ggacatttttctttgaattagagcctcagcagctcgtcattgctgaattttcttgaagt (SEQ ID NO: 1)

Where site 10 confers the trait of oil metabolization and can be (A) or (T), site 20 confers optimal growth in acidic waters and can be (T) or (C), and site 30 confers resistance to penicillin and can be (A) or (G).

These sites are then represented as a vector, where:
α1 rs10 (A) (this genotype is represented as the vector α1 (1, 0) (If it were (T) it would be α1 (0, 1)
α2 rs20 (T) (this genotype is represented as the vector α2 (0, 1) (If it were (C) it would be α1 (1, 0)
α3 rs30 (A) (this genotype is represented as the vector α3 (0, 1) (If it were (G) it would be α1 (1, 0)

This series of markers is then concatenated into the vector: F<α1, α2, α3>which is notated as Γ1<1, 0, 0, 1, 0, 1>

In some embodiments, there are more than three genotypes which would make r<α1, α2, α3, α4, . . . > and in some embodiments, there are less than three genotypes.

In some embodiments, a neural network is used to generate fitness values for given genetic permutations in specific environments.

The gamma vector, which is specific to the genotype listed by the recipient, is then applied to a vector which has assigned values for the fitness of each genotype within each specific environmental parameter and preference parameter listed by the recipient with the following equation:

Γ·εsum=F for Genetics X Environment=Fitness

To find εsum, which is the sum of fitness values given for all parameters including recipient specified and non-specified environments, we have a set of premade weights for each specific environment and its genetic permutation.

εsum=ε1+ε2+ε3 . . .

Therefore εsum is a composite function of previously derived individual EN values (i.e. ε1, ε2, ε3, etc.). In some embodiments there are more vectors of EN values and in some embodiments, there are fewer.

In this example, ε1 is the vector denoting the fitness values for each genetic permutation according to the recipient's specification of oil metabolism in an oil-rich environment, ε2 is the vector denoting fitness values for each genetic permutation according to the recipient's desire for optimal growth in acidic waters, and ε3 is the vector denoting fitness values for each genetic permutation according to the recipient's preference for penicillin resistance.

Oil metabolism selectedε1<100,−10,−10,0,0,0>

ε1 has specific values corresponding to each genetic variation. Here, we show that the presence of oil confers a fitness disadvantage to those genomes which have the genetic permutation of (T) for rs20/vector α2. The fitness is not only affected by the ability to metabolize oil, but that there is a disadvantage for bacteria that have the trait that confers optimal growth in acidic conditions to be in an oil-rich environment, shown with the −10 value for those genomes with rs20/vector α2

Next, our recipient has indicated they would like an organism best suited for growth in an acidic environment, so we have the vector pertaining to acidic environments in respect to these genetic permutations as:

£2<0,0,50,0,0,0>

where we see a significant fitness value advantage for the permutation conferring oil metabolism in the oil-rich environment.

Next, ε3 accounts for the recipient preference of penicillin resistance.

ε3<0,0,0,0,0,100>

Here, we see a high relative fitness value assigned to the vector position accounting for penicillin resistance.

In some embodiments, the values for a specific genetic permutation are taken from the growth curve Y=Ae^{kx}, where the exponential value of proliferation in a given environment for an organism with given genetic permutations, is used as the values in the environmental vectors.

The calculations and steps are then followed as in example 2 to optimize the genome and deliver the genome to the recipient with the relevant modifications:
actttcaaaTaaatcagcgCaaaaaacatGctaatttgggcaaattcccacctgtttttag ggacatttttctttgaattagagcctcagcagctcgtcattgctgaattttcttgaagt (SEQ ID NO: 1)

If the recipient chooses, this sequence can be compared to existing strains of bacteria. In this hypothetical example, we compare this genome to the genomes that exist to find an existing strain with the highest percentage of congruence pertaining to the permutations desired;

Existing Strains:

Oceanobacillus Iheyensis Pacificus—66.7% congruence for desired traits (2/3)
actttcaaaAaaatcagcgCaaaaaacatGctaatttgggcaaattcccacctgtttttag ggacatttttctttgaattagagcctcagcagctcgtcattgctgaattttcttgaagt (SEQ ID NO: 2)

Oceanobacillus Iheyensis Atlanticus—33.3% congruence for desired traits (1/3)
actttcaaaTaaatcagcgTaaaaaacatActaatttgggcaaattcccacctgtttttag ggacatttttctttgaattagagcctcagcagctcgtcattgctgaattttcttgaagt (SEQ ID NO: 3)

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A system for genetic customization comprising:
one or more processors wherein said processors are configured to:
receive a specification including an environmental parameter or phenotype of interest;
receive a genotype of an organism to be modified;
determine genetic permutations pertaining to the phenotype of interest based at least in part on optimal fitness according to environmental parameters, recipient specifications, and loci of disease; and
deliver the optimized genotype to the recipient; and
a memory coupled to the processor, configured to provide the processor with instructions.

2. The system of claim 1, further comprising a database coupled to the processor, configured to store the genetic permutations.

3. The system of claim 1, wherein determining the fitness level of a given genotype includes determining all possible genomes and applying them to selected environmental parameters and specifications, which may or may include vectors considering traits that may cause illness.

4. The system of claim 1, wherein determining the fitness level of a given genotype includes applying all possible combinations of genetic permutations to recipient selected environmental parameters and specifications, which may include traits that may cause illness.

5. The system of claim 1, where the processor is further configured to identify a strain among the plurality of strains most congruent with the recipient specifications, based at least in part on the percentage match determined.

6. The system of claim 1, wherein the specification includes a plurality of independent environmental parameters.

7. The system of claim 1, wherein the processor is further configured to identify any strain that is deemed to be a close percentage match of the optimized genome.

8. The system of claim 1, wherein the processor is further configured to:
receive an additional specification or specifications that includes an additional phenotype of interest; and
determine additional genetic permutations pertaining to the additional phenotype of interest.

9. The system of claim 8, wherein the processor is further configured to supply display information for displaying the additional genetic permutations relevant to the phenotype(s) of interest.

10. A method for genetic customization comprising:
receiving a specification including an environmental parameter or phenotype of interest;
receiving a genotype of an organism to be modified;
calculating fitness pertaining to selected environmental parameters, recipient preference, and loci of disease;
determining genetic permutations pertaining to the phenotype of interest based at least in part on optimal fitness according to environmental parameters, recipient specifications, and loci of disease; and
delivering the optimized genotype to the recipient.

11. The method of claim 10, further comprising storing the genetic permutations of the plurality of environmental parameters, recipient specifications, and loci of disease.

12. The method of claim 10, wherein determining the genetic permutations pertaining to the optimized genome is further based on calculation of fitness.

13. The method of claim 10, wherein determining the fitness level of a given organism includes determining all possible combinations of genetic permutations.

14. The method of claim 10, wherein the recipient specifications are any combination of environmental parameters, phenotypes, and loci of disease, and determining the optimal genome accounts for the specifications.

15. The method of claim 10, further comprising excluding any donor that is deemed to be a close relative of the recipient.

16. The method of claim 10, further comprising: receiving an additional specification that includes an additional phenotype of interest; and determining additional statistical information pertaining to the additional phenotype of interest based at least in part on an additional genotype of the recipient and an additional genotype of the preferred donor.

17. The method of claim 12, wherein determining the genetic permutations pertaining to the optimized genome is further based on calculation of fitness is done by use of a neural network.

18. A computer program product for genetic customization, the computer program product being embodied in a computer readable storage medium and comprising computer instructions for:
receiving a specification including a phenotype of interest;
receiving a genotype of an organism and a plurality of genotypes of existing species;
determining fitness levels of all possible genomes based at least in part on recipient specifications including environmental parameters, loci of disease, and recipient phenotype preferences; and
determining percentage congruence pertaining to the genotype of interest vs. existing
strains based on environmental parameters and/or recipient specifications.
